(19) United States
(12) Patent Application Publication
(10) Pub. No.: US 2017/0164877 A1
(43) Pub. Date: Jun. 15, 2017

(54) MANUAL COMPUTER INTERFACE WITH INTEGRAL GLUCOSE MONITOR SYSTEM

(71) Applicant: Ronald Smith, Marysville, CA (US)

(72) Inventor: Ronald Smith, Marysville, CA (US)

(21) Appl. No.: 14/964,343

(22) Filed: Dec. 9, 2015

(51) Int. Cl. A61B 5/145; G06F 3/0354; A61B 5/00

(52) U.S. Cl. CPC A61B 5/14532; A61B 5/6897; G06F 3/03543; A61B 5/150358

(57) ABSTRACT

A glucose monitor computer mouse for enabling blood sugar testing from a coupled computer and automatically storing results on the coupled computer. The glucose monitor computer mouse includes both a computer mouse assembly, defining a conventional computer pointing device, and an integrated blood glucose meter which provides blood sugar readings from disposable testing strips like conventional blood glucose meters. The monitor computer mouse may operate with a tracking software application loaded on the coupled computer which automatically stores results and may automatically generate alerts when results are out of a desired range.

### BACKGROUND OF THE INVENTION

Field of the Invention

This invention relates generally to a combined glucose monitor and, more particularly, to a computer mouse having an integral glucose monitor and method for treating glucose levels.

Description of the Prior Art

The use of conventional glucose monitors for allowing the blood sugar level of a patient or user to be checked at desired times is well known. While glucose monitors are often employed by medical personnel in hospital or doctors' offices, home testing kits designed to allow a user to check their own levels have become more common in recent times. Despite the convenience offered by such home testing kits, a problem which still exists is that such kits commonly require a user to manually record the results of the blood sugar test manually. Moreover, while the portability of home testing kits is generally desirable, such portability leads to the kit being susceptible to being lost.

Thus, there remains a need for a manual computer interface with integral glucose monitor which allows a user to check their blood sugar while at their computer. It would be helpful if such a manual computer interface with integral glucose monitor was embodied as a computer mouse having an integrated blood glucose meter to provide an interface for conventional blood glucose test strips. It would be additionally desirable for such a manual computer interface with integral glucose monitor to automatically communicate testing results to a computer that the manual computer interface was electronically connected to.

The Applicant's invention described herein provides for a glucose monitor computer mouse adapted to receive and process blood glucose testing strips. The primary components in Applicant's glucose monitor computer mouse are a mouse assembly and an integral digital glucose meter. When in operation, the glucose monitor computer mouse enables more convenient testing of blood sugar levels and efficient recording of the same. As a result, many of the limitations imposed by prior art structures are removed.

### SUMMARY OF THE INVENTION

A glucose monitor computer mouse for enabling blood sugar testing from a coupled computer and automatically storing results on the coupled computer. The glucose monitor computer mouse includes both a computer mouse assembly, defining a conventional computer pointing device, and an integrated blood glucose meter which provides blood sugar readings from disposable testing strips like conventional blood glucose meters. The monitor computer mouse may operate with a tracking software application loaded on the coupled computer which automatically stores results and may automatically generate alerts when results are out of a desired range.

It is an object of this invention to provide a manual computer interface with integral glucose monitor which allows a user to check their blood sugar while at their computer.

It is another object of this invention to provide a manual computer interface with integral glucose monitor embodied as a computer mouse having an integrated blood glucose meter to provide an interface for conventional blood glucose test strips.

It is yet another object of this invention to provide a manual computer interface with integral glucose monitor which automatically communicates testing results to a computer that the manual computer interface was electronically connected to.

These and other objects will be apparent to one of skill in the art.

### BRIEF DESCRIPTION OF THE DRAWINGS

FIG. 1 is a top plan view of a glucose monitor computer mouse built in accordance with the present invention with a conventional test strip adjacent thereto.

FIG. 2 is a side elevational view of a glucose monitor computer mouse built in accordance with the present invention with a conventional test strip inserted therein.

FIG. 3 is a front elevational view of a glucose monitor computer mouse built in accordance with the present invention.

### DETAILED DESCRIPTION OF THE INVENTION

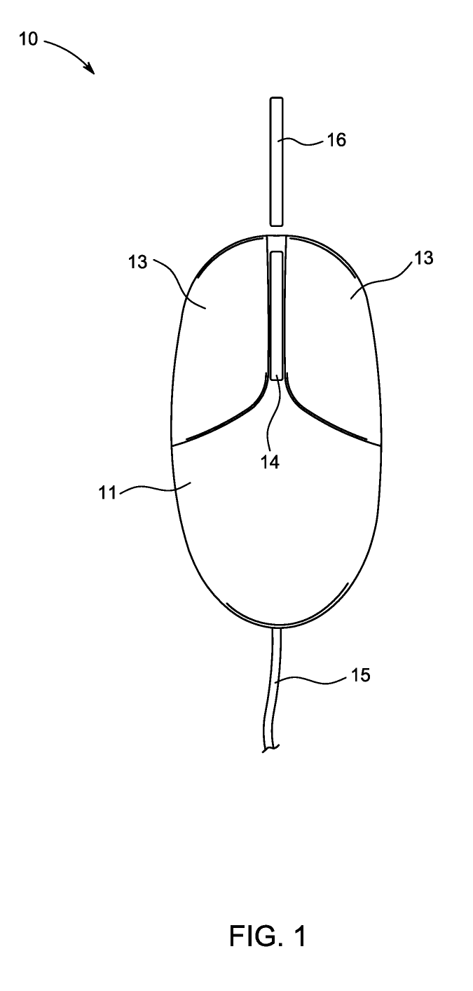

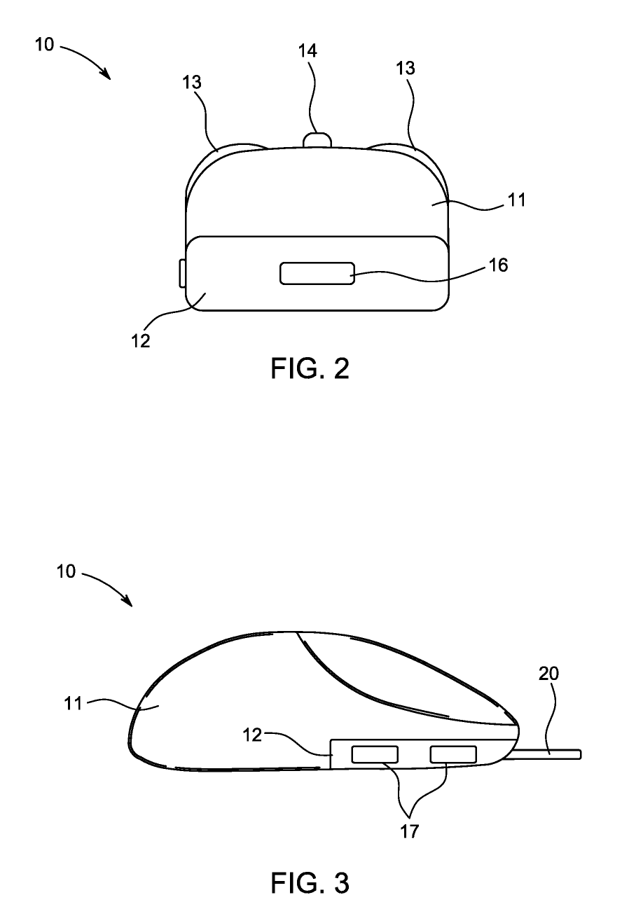

Referring now to the drawings and in particular FIGS. 1, 2, and 3, a glucose monitor computer mouse 10 is shown as one embodiment of a manual computer interface with integral glucose monitor in accordance with the present invention. The glucose monitor computer mouse 10 defining a computer mouse assembly 11 and an integrated blood glucose meter 12. The mouse assembly 11 defines a conventional electronic pointing device that detects physical two-dimensional motion relative to a surface and translates such physical motion to motion of a pointer on an electronic display so as to allow for fine control of a graphical user interface. In the some embodiments, the mouse assembly 11 includes two buttons 13 and a digital scroll wheel 14 as input interfaces. It is contemplated, however, that in other embodiments, the mouse assembly 11 may have any number of conventional mouse input interfaces. In addition, it is contemplated that the mouse assembly 11 may include a connection wire 15 to allow it to be coupled with a conventional computer (not shown) or may include an internal wireless antenna (not shown) 15 to allow it to be coupled with a conventional computer.

It is contemplated that the mouse assembly 11 is powered by the conventional electrical powering mechanisms of a conventional computer mouse. For example, a wired mouse assembly 11 may receive electricity from a connected computer through the wire 15 and a wireless mouse may include an onboard power source, such as a battery.

The blood glucose meter 12 is built into the monitor computer mouse 10 and operative to provide testing results as blood sugar readings as conventional blood glucose meters. The blood glucose meter 12 electrically coupled with the mouse assembly 11, thereby enabling the meter 12 to share the same electrical power source as used by the mouse assembly 11 as well as communicate testing results directly to the coupled computer. The meter 12 includes a test strip interface 16 which allows it to receive and electrically interface with a conventional disposable blood glucose test strip 20. The meter 12 additionally includes two interface buttons 17 which provide for manual control of the meter 12. It is appreciated that as the testing results are communicated directly to a coupled computer in the normal operation of the meter 12 obviates the need for the inclusion of a display screen on the meter 12. In some embodiments, however, a display screen for showing blood sugar readings is included monitor computer mouse 10.

In some embodiments, the buttons 17 are disposed on opposing sides of the monitor computer mouse 10.

It is contemplated that the monitor computer mouse 10 may operate with a tracking software application which is loaded on a coupled computer. When the tracking software application is loaded and detected by the meter 12, testing results are automatically stored through the application in tabular form. It is appreciated that such results may also be automatically transmitted by the coupled computer to medical personnel. In addition, the application may all for emergency alerts to be generated and transmitted when a single blood sugar reading or consecutive blood sugar readings are out of a preset desired range.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A glucose monitor computer mouse, comprising:
a single mouse housing adapted to be held in one hand by a user;
a computer mouse assembly defining a conventional electronic pointing device configured to be coupled with a conventional computer, wherein said mouse assembly is contained in said housing; and
a blood glucose meter operative to receive conventional disposable blood sugar testing strips and produce blood sugar readings therefrom, wherein said meter is contained in said housing and electrically coupled with said mouse assembly so as to enable the meter to utilize a power source available to the mouse assembly and communicate electrical signals to the conventional computer to which the mouse assembly is connected.

2. The glucose monitor computer mouse of claim 1, additionally comprising a test strip interface which adapts the computer mouse to receive and electrically interface with a conventional disposable blood glucose test strip.

3. The glucose monitor computer mouse of claim 2, wherein said mouse assembly includes a wire which configures the computer mouse assembly to be coupled with the conventional computer and said wire and said test strip interface are disposed on opposing ends of said computer mouse.

4. The glucose monitor computer mouse of claim 1, wherein said blood glucose meter is operative to automatically communicate electrical signals which define blood sugar readings to the conventional computer to which the mouse assembly is connected upon generating blood sugar readings from blood sugar testing strips.

5. The glucose monitor computer mouse of claim 4, wherein said conventional computer is configured to automatically store blood sugar readings upon receiving blood sugar readings from the blood glucose meter.

6. The glucose monitor computer mouse of claim 4, wherein said conventional computer is configured to selectively generate an emergency alert based on blood sugar readings received from the blood glucose meter.
